(19) United States
(12) Patent Application Publication
(10) Pub. No.: US 2026/0209661 A1
(43) Pub. Date: Jul. 23, 2026

(54) CELL CULTURE CONTAINER, CELL CULTURE DEVICE, AND CELL CULTURE METHOD

(71) Applicants: Hitachi, Ltd., Chiyoda-ku, Tokyo (JP); Tokai University Educational System, Shibuya-ku, Tokyo (JP)

(72) Inventors: Takayuki NOZAKI, Tokyo (JP); Masaharu KIYAMA, Tokyo (JP); Shizu TAKEDA, Tokyo (JP); Yuki NIIMI, Tokyo (JP); Kakuro HIRAI, Tokyo (JP); Hiroko HANZAWA, Tokyo (JP); Masato SATO, Isehara-shi, Kanagawa (JP); Miki KATO, Isehara-shi, Kanagawa (JP)

(21) Appl. No.: 19/134,894

(22) PCT Filed: Dec. 6, 2022
(86) PCT No.: PCT/JP2022/044858

§ 371 Date: Jun. 2, 2025

(51) Int. Cl. C12M 1/12; C12M 1/00; C12M 3/00

(52) U.S. Cl. CPC C12M 23/04; C12M 23/34; C12M 23/38; C12M 23/44; C12M 25/14; C12M 29/18

(57) ABSTRACT

Provided is a cell culture container that enables easy removal of a plurality of cell sheets cultured in one cell culture container. The cell culture container includes: a frame body that partitions the culture container; and a base member that is disposed on a bottom surface of the culture container for each of a plurality of sections partitioned by the frame body, in which the base member is removable for each of the sections.

### TECHNICAL FIELD

The present invention relates to a structure of a cell culture container, and a cell culture device and a cell culture method using the cell culture container.

### BACKGROUND ART

Regenerative medicine for recovering dysfunctional or damaged tissues using regenerated tissues or cells is expected as radical therapy for diseases for which there has been no conventional therapy. One of representative transplantation forms of the regenerative medicine includes a cell sheet in which cells are bound to each other or a cell and an extracellular matrix are bound to each other. Due to a demand for cell sheets expected in the future, there is a need to develop a technology for culturing and producing a plurality of cell sheets in large quantities in order to industrially supply the cell sheets with consistent quality.

Examples of a background art of the technical field include a technology such as PTL 1. PTL 1 discloses a method of repeating a process of culturing cells in a nest incorporated in a culture dish whose surface is coated with a temperature-responsive polymer to obtain a cell sheet, and collecting the cell sheet with a cultured cell transfer jig including a cell attachment portion.

### CITATION LIST

### Patent Literature

PTL 1: JP 2015-192640 A

### SUMMARY OF INVENTION

### Technical Problem

Meanwhile, in the case of automatically culturing cell sheets for a plurality of persons in a culture device, culturing the cell sheet for each patient using a closed-system culture container increases complexity of flow paths of the culture device. Therefore, it is necessary to produce cell sheets for a plurality of patients in a large-area culture container.

In the case of producing cell sheets for a plurality of patients at a time, it is necessary to peel off a large-area cell sheet from a bottom surface of the culture container.

In addition, it is necessary to cut the large-area cell sheet for each patient.

However, PTL 1 does not describe that the cell sheet adhering to the bottom surface of the culture container during culture is taken out without being peeled off from the bottom surface of the culture container. Therefore, it is considered that it takes time to peel off a plurality of cell sheets from the culture container.

Therefore, an object of the present invention is to provide a cell culture container in which a plurality of cell sheets cultured in one cell culture container can be easily removed, and a cell culture device and a cell culture method using the cell culture container.

### Solution to Problem

In order to solve the above problems, a cell culture container according to the present invention includes: a frame body that partitions the culture container; and a base member that is disposed on a bottom surface of the culture container for each of a plurality of sections partitioned by the frame body, in which the base member is removable for each of the sections.

A cell culture device according to the present invention includes: a cell culture container that includes a frame body that partitions the culture container, and a base member that is disposed on a bottom surface of the culture container for each of a plurality of sections partitioned by the frame body, the base member being removable for each of the sections, the frame body having a height smaller than a height of a side wall of the culture container; a lid that covers an opening of the culture container; a liquid holding portion that stores a culture solution; a liquid feeding pipe that penetrates through the lid and supplies the culture solution from the liquid holding portion to the culture container; and a discharge pipe that penetrates through the lid and discharges the culture solution from the culture container.

A cell culture method according to the present invention includes: supplying a culture solution containing cells to a culture container and culturing a cell sheet on a base member that is disposed in each of a plurality of sections partitioned by a frame body and is removable for each of the sections; removing the frame body; and acquiring the base member on which the cultured cell sheet is placed.

### Advantageous Effects of Invention

According to the present invention, it is possible to implement a cell culture container in which a plurality of cell sheets cultured in one cell culture container can be easily removed, and a cell culture device and a cell culture method using the cell culture container.

As a result, a process of taking out a cell sheet from a culture container becomes simpler, and the produced cell sheet can be efficiently transported to a transplantation site.

Problems, configurations, and effects other than those described above will become apparent by the following description of embodiments.

### BRIEF DESCRIPTION OF DRAWINGS

FIG. 1A is a plan view of a cell culture container according to a first embodiment of the present invention.

FIG. 1B is a cross-sectional view taken along line A-A′ of FIG. 1A.

FIG. 1C is an exploded view of the cell culture container in FIG. 1B.

FIG. 1D is a view illustrating a state in which a cell sheet is cultured.

FIG. 2A is a view illustrating a modified example of FIG. 1D.

FIG. 2B is an exploded view of the cell culture container in FIG. 2A.

FIG. 3A is a view illustrating a modified example of FIG. 1A.

FIG. 3B is a view illustrating a modified example of FIG. 1A.

FIG. 3C is a view illustrating a modified example of FIG. 1A.

FIG. 3D is a view illustrating a modified example of FIG. 1A.

FIG. 4A is a view illustrating one process of a cell culture method according to the first embodiment of the present invention.

FIG. 4B is a view illustrating a process subsequent to FIG. 4A.

FIG. 4C is a view illustrating a process subsequent to FIG. 4B.

FIG. 4D is a view illustrating a process subsequent to FIG. 4C.

FIG. 4E is a view illustrating a process subsequent to FIG. 4D.

FIG. 4F is a view illustrating a process subsequent to FIG. 4E.

FIG. 4G is a view illustrating a process subsequent to FIG. 4F.

FIG. 4H is a view illustrating a process subsequent to FIG. 4G.

FIG. 5 is a diagram illustrating a schematic configuration of a cell culture device according to the first embodiment of the present invention.

FIG. 6 is a diagram illustrating a configuration of a flow path of the cell culture device according to the first embodiment of the present invention.

FIG. 7A is a view illustrating a display example of a display screen 509 in FIG. 5.

FIG. 7B is a view illustrating a display example of the display screen 509 in FIG. 5.

FIG. 7C is a view illustrating a display example of the display screen 509 in FIG. 5.

FIG. 7D is a view illustrating a display example of the display screen 509 in FIG. 5.

FIG. 7E is a view illustrating a display example of the display screen 509 in FIG. 5.

FIG. 7F is a view illustrating a display example of the display screen 509 in FIG. 5.

FIG. 8 is a flowchart of the cell culture method according to the first embodiment of the present invention.

FIG. 9A is a view illustrating a cross-sectional shape of a frame body 901 according to a second embodiment of the present invention.

FIG. 9B is a view illustrating a modified example of FIG. 9A.

FIG. 9C is a view illustrating a modified example of FIG. 9A.

FIG. 9D is a view illustrating a modified example of FIG. 9A.

FIG. 9E is a view illustrating a modified example of FIG. 9A.

FIG. 10A is a plan view illustrating a state during culture in a cell culture container according to the second embodiment of the present invention.

FIG. 10B is a view illustrating a state in which one frame body 1002 is removed from FIG. 10A.

FIG. 10C is a view illustrating a state in which FIG. 10A is viewed from the side.

FIG. 10D is a view illustrating a state in which FIG. 10B is viewed from the side.

FIG. 10E is a view illustrating a state in which the frame body, a base member, a cell sheet, and a support are packaged.

FIG. 11A is a view illustrating a cell culture container according to a third embodiment of the present invention.

FIG. 11B is an exploded view of the cell culture container of FIG. 11A.

FIG. 11C is a view illustrating a modified example of FIG. 11A.

FIG. 11D is an exploded view of the cell culture container of FIG. 11C.

### DESCRIPTION OF EMBODIMENTS

Hereinafter, embodiments of the present invention will be described with reference to the drawings. In the drawings, the same components are denoted by the same reference numerals, and a detailed description of overlapping components is omitted.

### First Embodiment

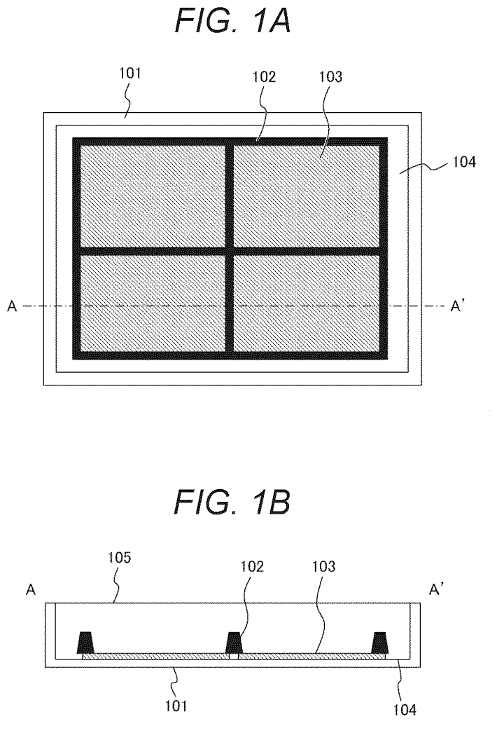

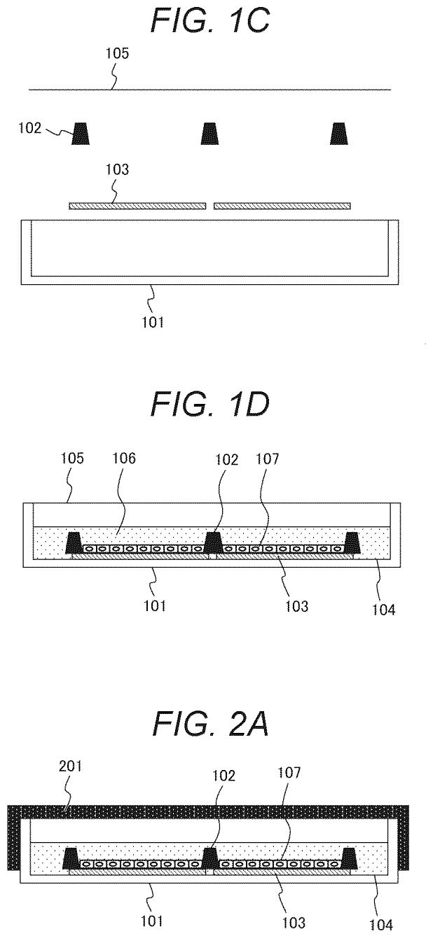

A cell culture container according to a first embodiment of the present invention, and a cell culture device and a cell culture method using the cell culture container will be described with reference to FIGS. 1A to 8.

First, a configuration of a cell culture container 101 according to the present embodiment will be described with reference to FIGS. 1A to 1D. FIG. 1A is a plan view of the cell culture container when viewed from above. FIG. 1B is a cross-sectional view taken along line A-A′ of FIG. 1A. FIG. 1C is an exploded view of the cell culture container in FIG. 1B. FIG. 1D is a view illustrating a state in which a cell sheet is cultured using the cell culture container 101.

FIGS. 1A to 1C illustrate a configuration of the cell culture container 101 and a removable culture member inside the cell culture container 101. The removable culture member includes a frame body 102 and a base member 103. The base member 103 enables adhesion and proliferation of cells. The frame body 102 is in contact with a peripheral edge of the base member 103 in a vertical direction.

With such a configuration, the peripheral edge of the base member 103 is placed under the frame body 102, and thus, a medium is present in the base member 103 during culture, and it is possible to avoid displacement or floating of the base member 103. In addition, since the cells do not grow on the peripheral edge of the base member 103 positioned under the frame body 102, the base member 103 can be taken out without damaging the cultured cells by culturing the cells, removing the frame body 102, and then gripping the peripheral edge of the base member 103 on which the cells do not grow. The base member 103 is disposed independently for each section surrounded by the frame body 102 and is not disposed across adjacent sections. The frame body 102 includes a material to which the cells do not adhere. Specifically, the frame body 102 is made of a material such as silicone and is produced by casting, for example. A surface of the frame body 102 is made non-cell-adhesive due to a property of the material itself such as silicone or surface treatment such as plasma treatment performed after the frame body 102 is produced. As a result, the cells are prevented from adhering to the frame body 102 during the culture. In addition, even in a case where the cells adhere to an outside portion 104 of the frame body 102, the cells do not move to other sections because the frame body 102 is non-cell-adhesive.

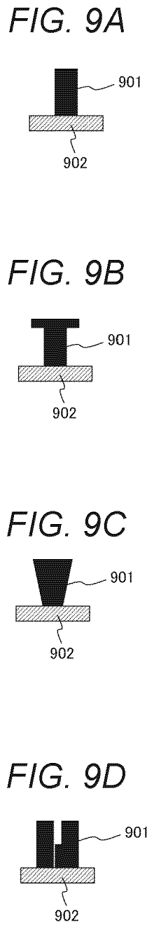

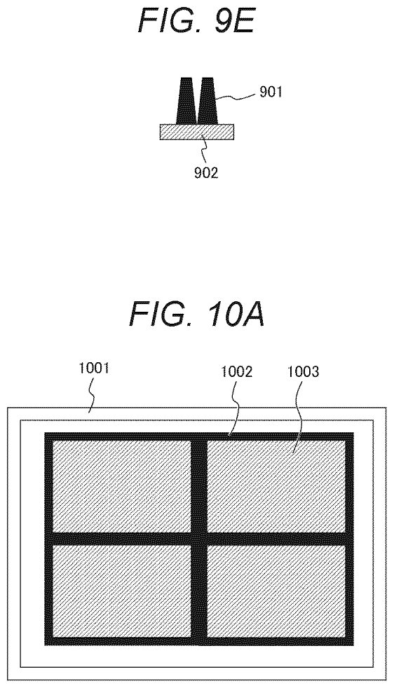

A cross-sectional shape of the frame body 102 is, for example, a trapezoidal shape as illustrated in FIG. 1B. The frame body 102 may have other cross-sectional shapes as described below in a second embodiment (FIGS. 9A to 9E). A height of the frame body 102 is, for example, about 1 mm, and is about 1000 times the cell diameter. As the frame body 102 has a certain height, it is possible to prevent movement of the cells between the sections formed by the frame body 102 even in a case where the cells adhere onto the frame body 102 and move on the frame body 102.

In addition, the height of the frame body 102 is set to such a height that movement of the medium is not hindered in agitation at the time of cell seeding or replacement of the medium described below. Specifically, the height of the frame body 102 may be smaller than a height of a side wall of the cell culture container 101, and the height of the frame body 102 may be about ½ of the height of the side wall of the cell culture container 101. In addition, an amount of the medium varies depending on a cell type. Specifically, a height of the medium may be set to several mm to several cm, and the amount of the medium may be sufficient to cover the frame body 102.

As illustrated in FIG. 1A, the frame body 102 forms one more sections in the cell culture container 101. A size of each section is the same as a size of the cell sheet used for transplantation. The base member 103 is placed in each section inside the frame body 102, and the cells are cultured on the base member 103.

The cell culture container 101 may be made non-cell-adhesive by the method described above. As a result, a bottom surface of the cell culture container 101 at the outside portion 104 of the frame body becomes non-cell-adhesive, and the cells are prevented from adhering to the outside portion 104 of the frame body.

It is sufficient if a base member whose size is larger than an area of each section inside the frame body 102 is installed as the base member 103, and an area of a portion of the base member 103 that is exposed when the base member 103 is pressed by the frame body 102 may be the same as the size of the cell sheet used for transplantation.

The base member 103 is desirably a thin film having porosity. It is sufficient if a material of the base member 103 is plastic, and specific examples of the material include urethane acrylate, polyester acrylate, epoxy acrylate, polymethyl (meth)acrylate, ethoxylated bisphenol A acrylate, aliphatic urethane acrylate, polyester acrylate, polyethylene terephthalate, polystyrene, polycarbonate, acrylic-modified alicyclic epoxide, bifunctional alcohol ether type epoxide, acrylic silicone, and acrylic dimethylsiloxane. In addition, the material may be fibrin glue or Beriplast which is a tissue adhesive.

As illustrated in FIG. 1C, the base member 103 is placed on the cell culture container 101, and then the frame body 102 is placed. The base member 103 may be attached to the frame body 102 in advance by means such as thermal welding. In this case, it is desirable to attach the base member 103 with such a strength that the base member 103 can be easily removed from the frame body 102 after the culture by a method such as gripping the base member 103 with tweezers or the like. The base member 103 may be firmly attached and cut with a scalpel or the like.

In order to prevent biological contamination due to bacteria and the like entering from the outside of the cell culture container 101 during the culture in a state in which the frame body 102 and the base member 103 are installed in the cell culture container 101, a lid 105 that covers the entire opening of the cell culture container 101 is installed on the cell culture container 101. The lid 105 may be made of, for example, the same material as that of the cell culture container 101, and may be installed by a method such as thermal welding. Alternatively, the lid 105 may be installed by screwing or the like.

The cell culture container 101 may include a medium supply pipe, a discharge pipe, a gas feeding pipe, and the like according to a form of the cell culture device, and it is sufficient if each pipe is installed according to a medium replacement method and a gas exchange method. The pipes are installed so as to penetrate through the lid 105. Since the cell culture container 101 enables production of a plurality of cell sheets in one cell culture container 101, it is not necessary to provide a plurality of supply pipes, discharge pipes, and gas feeding pipes in the cell culture device.

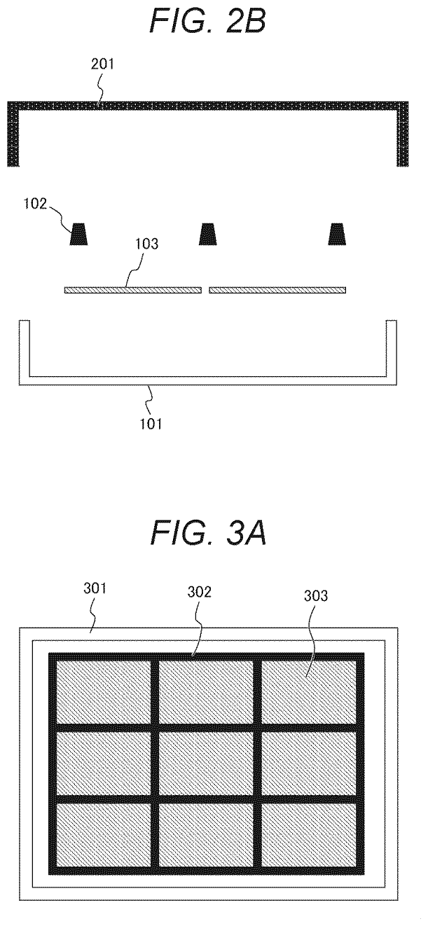

FIGS. 2A and 2B illustrate modified examples. FIG. 2A is a view illustrating a modified example of FIG. 1D, and FIG. 2B is an exploded view of the cell culture container in FIG. 2A.

As illustrated in FIGS. 2A and 2B, a lid 201 that can be easily opened and closed may be provided instead of the lid 105. A longitudinal section of the lid 201 has a recessed shape, and the lid 201 is molded such that an inner side surface of the lid 201 is fitted to an outer side surface of the cell culture container 101 when the lid 201 is put on the cell culture container 101. The lid 201 can be easily opened and closed by gripping an outer side surface of the lid 201. As the lid 201 has such a shape, the lid 201 can be opened and closed aseptically in a safety cabinet or the like, and necessary work can be performed when performing operations such as cell seeding and medium replacement.

As illustrated in FIG. 1D, at the time of the culture, when the cells are seeded together with a medium 106, the cells that have reached the base member 103 in each section inside the frame body 102 adhere, extend, and proliferate until the cells form a sheet after the culture, thereby forming a cell sheet 107. Since the frame body 102 is non-cell-adhesive, there is no cell on the frame body 102.

In a case where the outside portion 104 of the frame body is non-cell-adhesive, cells that have reached the outside portion 104 of the frame body are removed at the time of medium replacement. In a case where the outside portion 104 of the frame body is cell-adhesive, the cells adhere, extend, and proliferate, but do not move to each section inside the frame body 102 because the frame body 102 is non-cell-adhesive.

After the culture, the lid 105 is aseptically removed, and the frame body 102 is aseptically taken out in the same manner. Then, the base member 103 is taken out. Since the base member 103 is merely placed on the cell culture container 101, the base member 103 can be easily taken out from the cell culture container 101 by a method such as gripping with tweezers. That is, the cultured cell sheets 107 can be easily taken out one by one from the cell culture container 101.

In addition, since the base member 103 is partitioned by the frame body 102 in the same size as the cell sheet used for transplantation, it is not necessary to cut the cell sheet after the culture. One base member may be for one patient, or a plurality of base members may be for one patient.

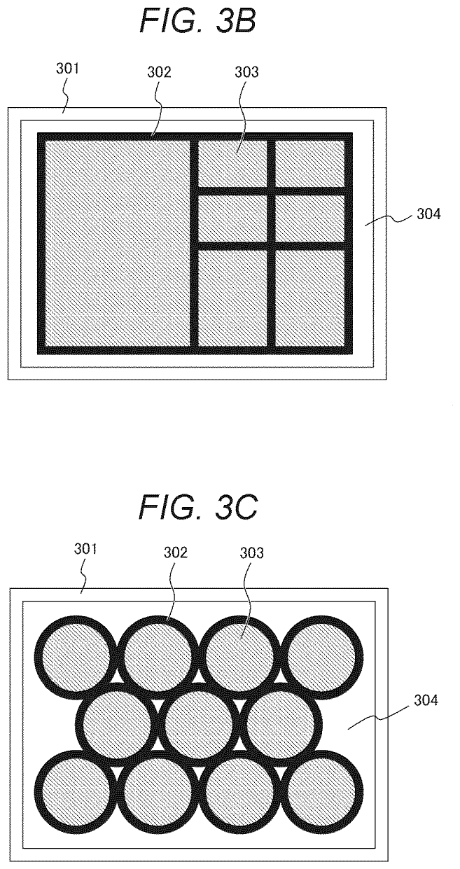

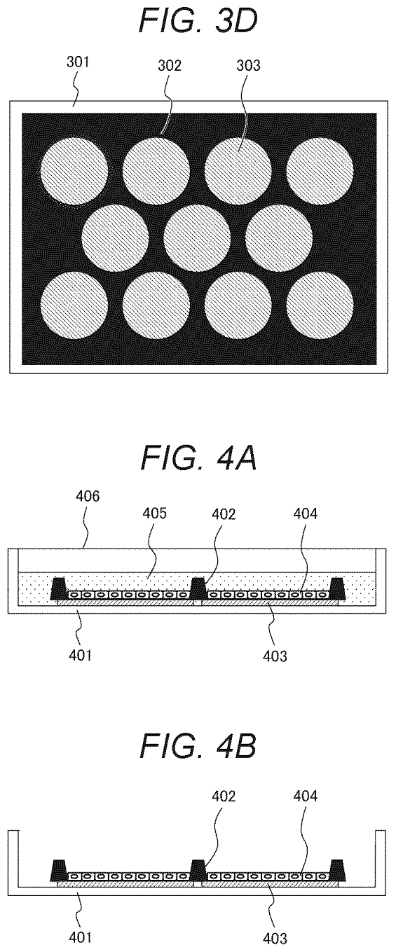

FIGS. 3A to 3D all illustrate modified examples of FIG. 1A and are views illustrating examples of shapes of the respective sections inside the frame body 102 of the cell culture container 101 described in FIGS. 1A to 1D. In FIG. 3A, the area of each section in FIG. 1A is reduced by a frame body 302. In FIG. 3B, one cell culture container 301 has sections having a plurality of different sizes by the frame body 302. FIG. 3B illustrates sections of three sizes, large, medium, and small. In FIGS. 3C and 3D, the shape of the section defined by the frame body 302 is a circle. FIG. 3C illustrates an example in which an outside portion 304 of the frame body is provided, and FIG. 3D illustrates an example in which an area of the frame body 302 is large and the outside portion 304 of the frame body is not provided.

The size and shape of the section may be appropriately selected according to a size and shape of a transplant site to be transplanted. The shape of the section may be a shape other than a circle. It is possible to produce a cell sheet having a desired size and shape, and it is possible to omit a cell sheet cutting process that requires skilled work.

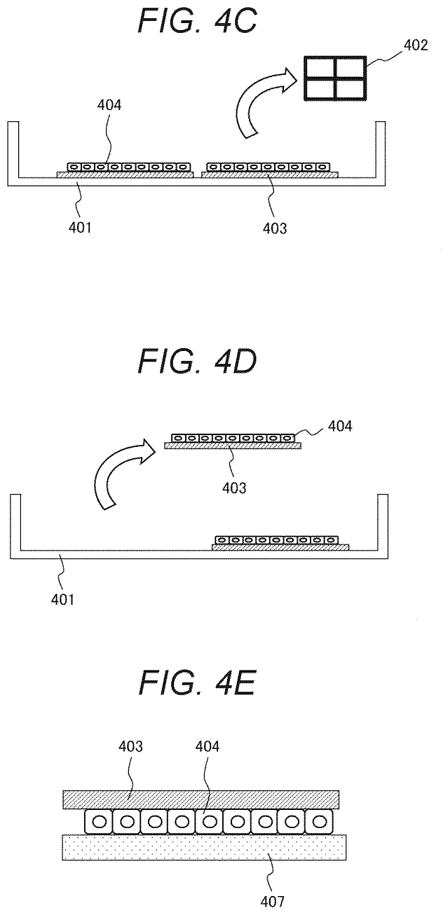

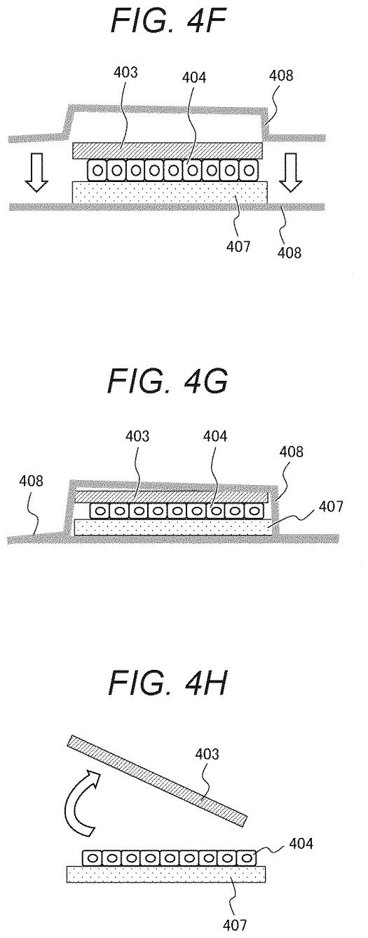

FIGS. 4A to 4H are views illustrating processes of culture, collection, and packaging of a cell sheet in a cell culture container.

FIG. 4A illustrates a state immediately after culture. A frame body 402 and a base member 403 are installed inside a cell culture container 401, and a cell sheet 404 adheres onto the base member 403. A medium 405 is placed in the cell culture container 401. After the culture, the medium 405 is discharged and washed with physiological saline, phosphate-buffered saline (PBS), or the like an appropriate number of times. The washing work may be automatically performed using a closed-system cell culture device, or may be manually performed in a safety cabinet by removing the cell culture container 401 from the closed-system cell culture device.

Subsequently, a lid 406 is removed as illustrated in FIG. 4B. In a case where the above-described washing is manually performed in the safety cabinet, the washing may be performed after removing the lid 406.

Next, as illustrated in FIG. 4C, the frame body 402 is gripped with tweezers or the like and aseptically removed. Since the frame body 402 is non-cell-adhesive, only the frame body 402 can be removed without pulling up cells on the base member 403. In a case where the frame body 402 is cell-adhesive, the cell sheet 404 on the base member 403 and the frame body 402 are connected via the cells, and the cell sheet 404 also moves when the frame body 402 is lifted. In order to avoid such a situation, the frame body 402 needs to be non-cell-adhesive.

Next, as illustrated in FIG. 4D, the base member 403 to which the cell sheet 404 adheres is gripped with tweezers or the like and aseptically removed from the cell culture container 401.

Next, as illustrated in FIG. 4E, the base member 403 and the cell sheet 404 are placed on a support 407 prepared in advance. At this time, the cell sheet 404 is placed so as to be in contact with the support 407. The support 407 may be a hydrophilic polyvinylidene difluoride (PVDF) membrane, a nitrocellulose membrane, or the like in addition to a support made of fibrin glue or Beriplast which is a tissue adhesive.

Then, as illustrated in FIG. 4F, the support 407 is placed on a packaging member 408, and another packaging member 408 is put on the base member 403, the cell sheet 404, and the support 407 so as to cover the base member 403, the cell sheet 404, and the support 407.

Next, as illustrated in FIG. 4G, the upper and lower packaging members 408 are thermally welded, so that the base member 403, the cell sheet 404, and the support 407 are sealed with the packaging members 408. In this state, the cell sheet 404 is transported to an operating room in a state in which a temperature, a pressure, and cleanliness are appropriately maintained. In order to maintain a quality of the cells at the time of the transportation, the cells may be transported in a state in which a transportation solution is put in the packaging member 408 by using a transportation solution inlet (not illustrated) and a transportation solution outlet (not illustrated) attached to the packaging member 408.

Since a process of peeling off the cell sheet 404 from the base member 403 is not performed before the transportation, a time required for treatment of the cell sheet 404 can be reduced, and an influence of the atmosphere on the cell sheet 404 can be minimized.

After the transportation, the cell sheet 404, the base member 403, and the support 407 are aseptically taken out from the packaging member 408, and the base member 403 is peeled off from the cell sheet 404 as illustrated in FIG. 4H. For example, the base member 403 is gripped with tweezers or the like and peeled off from the cell sheet 404.

The base member 403 and the cell sheet 404 may be subjected to, for example, treatment using enzyme such as dispase to decompose only a basal layer of the cell sheet 404, and then the base member 403 may be gripped with tweezers or the like and removed as illustrated in FIG. 4H. In addition, the base member 403 may be used as a temperature-responsive culture surface, treated in a hydrophobic and cell-adhesive state during the culture, and treated in a hydrophilic and non-cell-adhesive state by lowering the temperature after the transportation, and the base member 403 is gripped with tweezers or the like and removed as illustrated in FIG. 4H.

In addition, a tissue adhesive in a state before solidification is placed as the support 407 on the cell sheet 404, the cell sheet 404 and the tissue adhesive are firmly integrated with each other by solidifying the tissue adhesive, and then the support 407 which is the tissue adhesive may be lifted with tweezers to peel off the cell sheet 404 from the base member 403. Since the cell sheet 404 and the tissue adhesive are firmly integrated with each other, the cell sheet 404 is easily peeled off from the base member 403. In a case where the support 407 is a tissue adhesive, the support 407 can also be transplanted.

In a case where the support 407 is a hydrophilic PVDF membrane, a nitrocellulose membrane, or the like, the hydrophilic PVDF membrane, the nitrocellulose membrane, or the like is removed after the transplantation.

The cell sheet 404 is packaged without removing the base member 403 and then transported to the operating room as illustrated in FIGS. 4F and 4G. However, the cell sheet 404 may also be packaged in a state in which the base member 403 is removed, and then transported to the operating room. In this case, the base member 403 is removed before the cell sheet 404 is packaged, and the cell sheet 404 is transported in a state in which the base member 403 is peeled off, so that work in the operating room can be reduced.

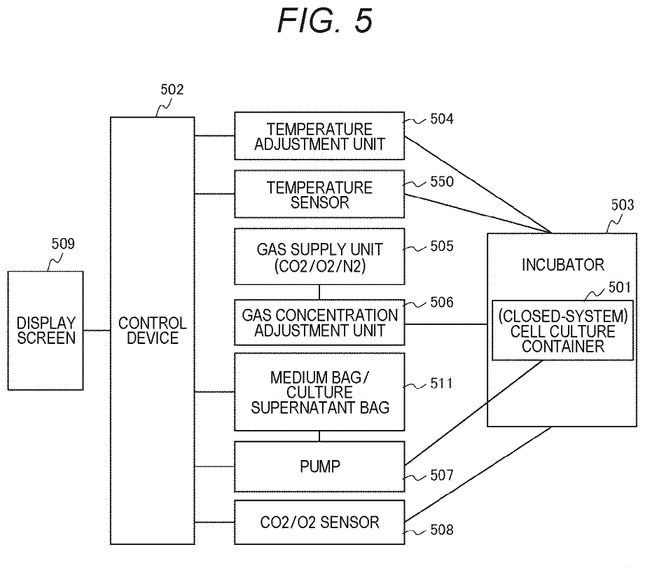

With the cell culture container configured as described above, the cultured cell sheets can be taken out one by one from the cell culture container without being peeled off from the base member, so that the taking out of the cell sheet can be simplified. In addition, when the cell sheet is taken out from the cell culture container, the cell sheet can be taken out without preparing an additional device or the like. FIG. 5 is a diagram illustrating a schematic configuration of the cell culture device according to the present embodiment. The cell culture container according to the present embodiment can be used for both a closed-system cell culture device and a housing type cell culture device, but in the present embodiment, an example using a closed-system cell culture device is illustrated as an example.

As illustrated in FIG. 5, in a closed-system cell culture device including a closed-system cell culture container 501, each component controlled by a control device 502 is connected to a cell culture container 501 disposed inside an incubator 503. A temperature adjustment unit 504 for controlling a temperature of the incubator 503, a gas concentration adjustment unit 506 for controlling a gas concentration in the incubator 503, a pump 507 installed in a closed-system flow path circuit for automatically feeding a medium in the cell culture container 501, a CO2/O2 sensor 508, a medium bag/culture supernatant bag 511, and a temperature sensor 550 are connected to the control device 502. A gas supply unit 505 is connected to the gas concentration adjustment unit 506, and gas such as CO2, O2, or N2 is supplied from the gas supply unit 505 to the gas concentration adjustment unit 506.

The control device 502 corresponds to a processing unit and a storage unit in a normal computer including the processing unit including a central processing unit (CPU), the storage unit, an input/output unit including a display device and a keyboard, and a display screen 509 corresponds to a display unit of the display device.

The control device 502 operates various programs stored in the storage unit on the CPU as the processing unit. As a result, a culture environment in the incubator 503 is controlled by the temperature adjustment unit 504, the gas supply unit 505, the pump 507, the CO2/O2 sensor 508, the gas concentration adjustment unit 506, the temperature sensor 550, and the medium bag/culture supernatant bag 511, and a predetermined culture process in the cell culture container 501 can be performed.

Gas exchange in the cell culture container 501 is performed between the incubator 503 and the cell culture container 501 by a gas permeable membrane installed in the cell culture container 501. The gas concentration adjustment unit 506 may be directly connected to the cell culture container 501. The temperature adjustment unit 504, the gas concentration adjustment unit 506, and the CO2/O2 sensor 508 may be directly connected to the cell culture container 501. In such a configuration, the gas is directly supplied into the cell culture container 501.

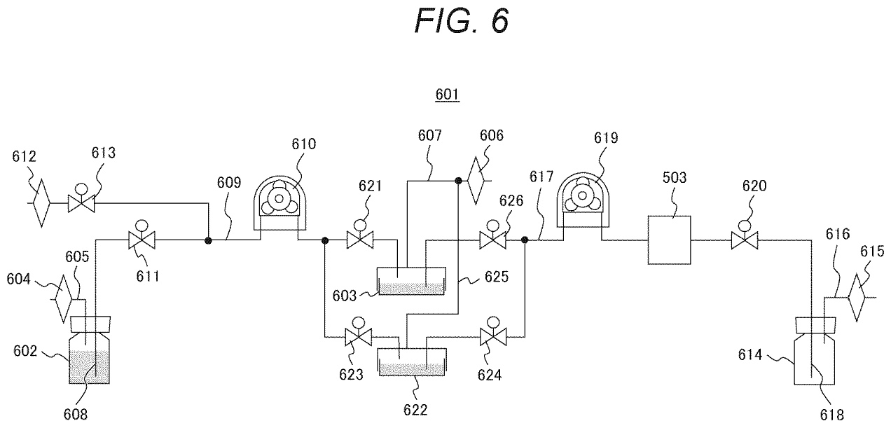

FIG. 6 is a diagram illustrating a configuration of a flow path of a cell culture device according to the present embodiment. FIG. 6 illustrates an example of a closed-system cell culture device having a closed-system flow path including a removable culture member.

A closed-system cell culture device 601 illustrated in FIG. 6 includes a first container 602 for storing a first liquid and a second container 603 for containing the first liquid. The first container 602 is a container for storing a medium for cell culture, the medium being the first liquid. The second container 603 is a container for culturing cells, and is a cell culture container.

The first container 602 and the second container 603 can be easily manufactured according to common technical knowledge of those skilled in the art in consideration of the purposes. The first container 602 includes an air pressure adjustment pipe 605 that is opened to the outside air via a filter 604, and a distal end of the air pressure adjustment pipe 605 is positioned in a gas phase inside the container. The second container 603 includes an air pressure adjustment pipe 607 that is opened to the outside air via a filter 606, and a distal end of the air pressure adjustment pipe 607 is positioned in a gas phase inside the container.

The closed-system cell culture device 601 includes a first liquid feeding pipe 608 for feeding the first liquid in the first container 602 and a second liquid feeding pipe 609 for feeding the first liquid in the first liquid feeding pipe 608 to the second container 603. The second liquid feeding pipe 609 includes a first liquid feeding pump 610 to adjust a liquid feeding amount in the second liquid feeding pipe 609. Each of the liquid feeding pipes 608 and 609 can be easily manufactured according to common technical knowledge of those skilled in the art. The first liquid feeding pipe 608 includes a first valve 611, and the first valve 611 can be opened and closed alternately with a second valve 613 that supplies air containing 5% CO2 through a filter 612 to switch between liquid feeding and gas feeding.

The closed-system cell culture device 601 also includes a third container 614 for discharging the first liquid in the second container (cell culture container) 603. The third container 614 can be easily manufactured according to common technical knowledge of those skilled in the art in consideration of the purpose. The third container 614 includes an air pressure adjustment pipe 616 that is opened to the outside air via a filter 615, and a distal end of the air pressure adjustment pipe 616 is positioned in a gas phase inside the container.

The closed-system cell culture device 601 further includes a first discharge pipe 617 for discharging the first liquid in the second container (cell culture container) 603 and a second discharge pipe 618 that is connected to the first discharge pipe 617 and discharges the first liquid in the second container (cell culture container) 603 to the third container 614 through the first discharge pipe 617. A second liquid feeding pump 619 controls a liquid feeding amount in the first discharge pipe 617. The liquid feeding pipes 608 and 609 and the discharge pipes 617 and 618 can be easily manufactured by common technical knowledge of those skilled in the art. The second discharge pipe 618 includes a third valve 620. The third valve 620 can be opened and closed to switch between enabling and disabling of the liquid feeding.

A plurality of containers can be connected in parallel to form the second container 603 which is the cell culture container. In the case of connecting the plurality of cell culture containers, it is sufficient if the second liquid feeding pipe 609 is provided with a branch, a fifth valve 621 is installed, and sixth valves 623 as many as the desired number of fourth containers 622 are provided. For the discharge of the first liquid, it is sufficient if the first discharge pipe 617 is provided with a branch, a seventh valve 626 is installed, and eighth valves 624 as many as the desired number of fourth containers 622 are provided. In order to adjust an internal pressure of the fourth container 622, it is sufficient if a branch for connection to the filter 606 and the same number of air pressure adjustment pipes 625 as the number of fourth containers 622 are installed.

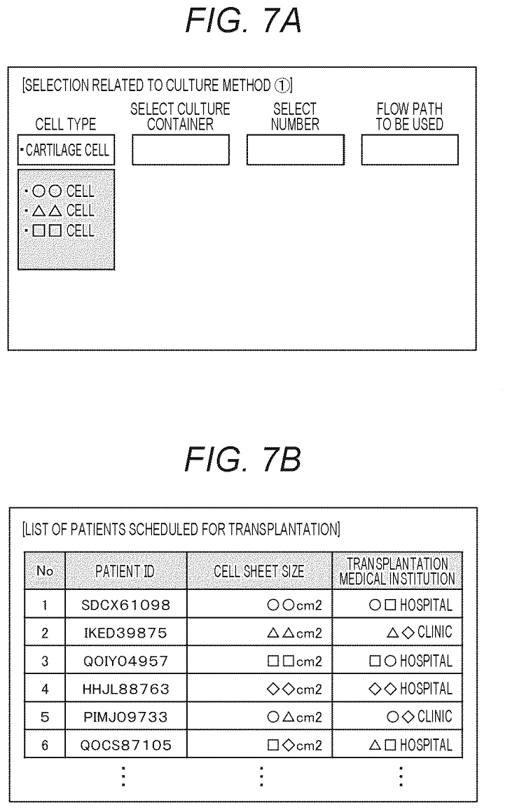

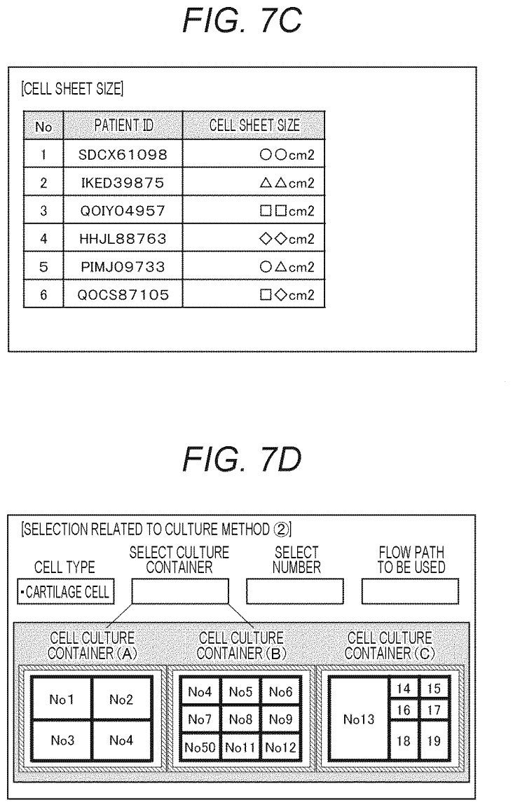

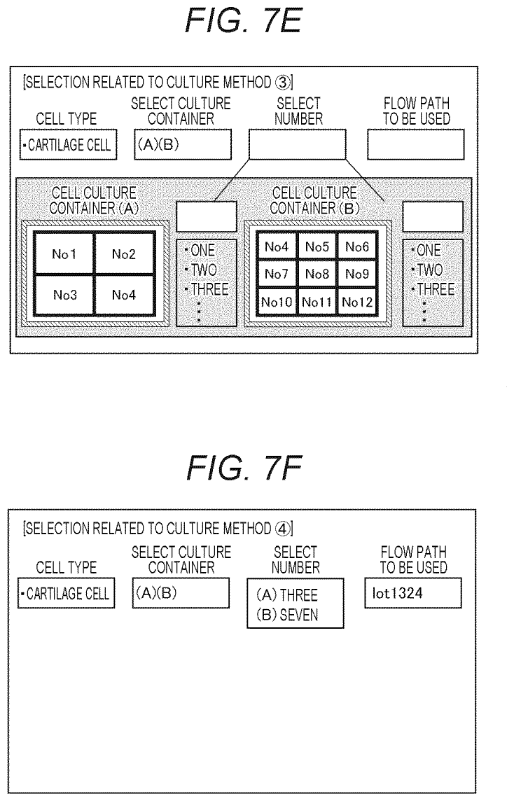

FIGS. 7A to 7F illustrate screen examples of a system of the closed-system cell culture device according to the present embodiment. Display examples of the display screen 509 of FIG. 5 are illustrated.

FIG. 7A illustrates a screen (1) for selection related to a culture method. As illustrated in FIG. 7A, the system of the closed-system cell culture device receives, as an input, information such as a cell type, the type of culture container, the number of culture containers, and a flow path to be used in the closed-system cell culture device. The system displays only selectable items among items to be selected subsequently, depending on a content of the previously selected item. As a result, it is possible to avoid a mismatch between a cell type to be cultured and a culture container or the like corresponding to the cell type. This function is similarly provided in the screens of FIG. 7B and subsequent drawings.

FIGS. 7B and 7C illustrate screen examples on which a list of patients to which each cell sheet is to be transplanted is displayed. FIG. 7B illustrates a list of patients scheduled for transplantation. Specifically, a patient number, a patient ID, a size of the cell sheet to be transplanted to each patient, a transplantation medical institution as a transportation destination, and the like are displayed. FIG. 7C illustrates the size of the cell sheet for each patient.

FIG. 7D illustrates a screen (2) for selection related to the culture method. The system of the closed-system cell culture device presents the type of culture container to be used as illustrated in FIG. 7D based on the size of the cell sheet required for transplantation presented in FIG. 7C. In addition, NO. of the patient associated with the cell sheet of each section is presented.

FIG. 7E illustrates a screen (3) for selection related to the culture method. After displaying FIG. 7D, the system of the closed-system cell culture device receives, as an input, the number of culture containers to be used as illustrated in FIG. 7E.

FIG. 7F illustrates a screen (4) for selection related to the culture method. As illustrated in FIG. 7F, the system of the closed-system cell culture device outputs information regarding the closed-system flow path used for automatic culture based on the input number of culture containers.

As described above, a medical institution to which the cell sheet is to be transported is determined according to patient information illustrated in FIG. 7 after the culture, and an identification code is assigned to each of the cell culture container, the frame body, the base member in the section of the frame body, a packaging, and a transportation container. The identification code is character information, a one-dimensional barcode, a two-dimensional barcode, a QR code, an RFID tag, or the like, and one or a plurality of identification codes are selected and assigned. For example, in a case where character information and an RFID tag are assigned, a human as an operator can recognize a content from the character information, and an electronic reader or the like can recognize the RFID tag, so that the content can be confirmed by two methods.

Information indicated by the identification code is information regarding a patient to which the cell sheet is to be transplanted, information regarding the cell sheet, information regarding a medical institution or the like to which the cell sheet is to be transported, an ID associated with these pieces of information, or the like. The identification code is used for management to prevent mix-ups at the time of peeling off the cultured cell sheet, packaging the cultured cell sheet, packing the cultured cell sheet into the transportation container, and transporting the cultured cell sheet. In addition, there are a case where the cell sheet is transported in a state in which the base member 403 is removed after the culture and a case where the cell sheet is transported without removing the base member 403, and both cases are reflected in the system of the closed-system cell culture device and are also reflected in a standard procedure manual referred to by the operator during the work. A type of the support 407 is a tissue adhesive, a hydrophilic PVDF membrane, a nitrocellulose membrane, or the like, and the type of the support 407 is also reflected in the system and the standard procedure manual.

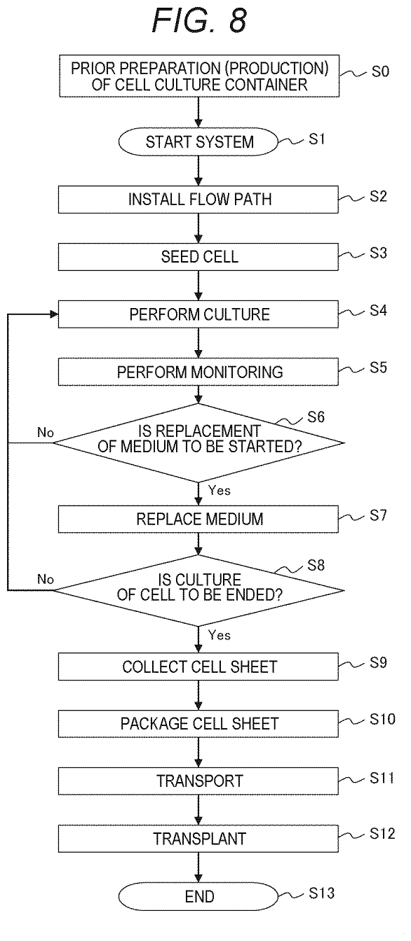

FIG. 8 illustrates a series of procedures for producing the cell sheet using the closed-system cell culture device having the above functions.

### Step S0: Prior Preparation of Cell Culture Container

First, as a prior preparation, the cell culture container is produced. Specifically, the cell culture container 101, the base member 103, the frame body 102, and the lid 105 are prepared as illustrated in FIG. 1C. One or more base members 103 are disposed at a bottom portion of the cell culture container 101, and the frame body 102 is further disposed at the peripheral edge of the base member 103. At this time, it is preferable to dispose the frame body 102 so as to press the peripheral edge of the base member 103 from above. This is to prevent the displacement and floating of the base member 103 as described above. Thereafter, the lid 105 covering the entire opening of the cell culture container 101 is installed on the cell culture container 101.

In this step, only the frame body 102 may be disposed at the bottom portion of the cell culture container 101, and the base member 103 may be disposed in each section partitioned by the frame body 102 after the flow path is installed in the subsequent step, for example, step S2. In this case, the lid 105 is installed after the base member 103 is placed in the cell culture container 101.

Thereafter, the produced cell culture container is sterilized. Examples of a sterilization method include γ-ray sterilization and EOG sterilization, and the sterilization method is appropriately selected based on a material and a surface of the used cell culture container. In a case where the closed-system flow path is connected to the cell culture container, the closed-system flow path is also sterilized.

### Step S1: Start of System

The system of the closed-system cell culture device is started. A device number or the like is described as information regarding the closed-system cell culture device to be used in a work procedure manual. An electronic tag or a two-dimensional barcode is installed in advance in the closed-system cell culture device, and the electronic tag or the two-dimensional barcode is read by a barcode reader or the like to confirm in advance that the information matches the information such as the device number described in the work procedure manual.

The operator presses and activates a start switch of an operation unit in the control device. Subsequently, the operator confirms whether or not an internal environment of the closed-system cell culture device is appropriate through an operation screen of a display (display screen 509) of a control unit. For example, the operator confirms that the temperature of the incubator is 37° C. The numerical values are not limited, and for example, the temperature can be selected from a range of 0° C. to 45° C. In addition, an automatic culture schedule is determined in advance by culture software, and the information is reflected on the closed-system cell culture device. The automatic culture schedule also includes conditions such as a date and time, a liquid amount, and the like when cell seeding, medium replacement, culture supernatant collection, test tissue collection, transplantation tissue collection, and the like are performed.

### Step S2: Flow Path Installation

The closed-system flow path including the cell culture container is installed in the closed-system cell culture device. According to the flow described in FIGS. 7A to 7F, the cell type, the type of cell culture container including the frame body having a predetermined number of sections, the number of cell culture containers, and the closed-system flow path to be used are determined in advance. In a case where only the frame body 102 is disposed at the bottom portion of the cell culture container 101 in step SO, the base member 103 is disposed in each section partitioned by the frame body 102.

When the closed-system flow path is carried out from a warehouse, an electronic tag or a two-dimensional barcode installed in advance in the closed-system flow path is read by a barcode reader or the like, thereby confirming in advance that the electronic tag or the two-dimensional barcode matches information such as the device number described in the work procedure manual. The closed-system flow path includes the cell culture container, the medium bag containing the medium, the culture supernatant bag for collecting a culture supernatant, and the like, and is implemented by a flow path tube connecting the cell culture container, the medium bag, the culture supernatant bag, and the like. A configuration of the closed-system flow path corresponds to the selected type of closed-system flow path. An identification code of the closed-system flow path also includes those for the cell culture container, the frame body, and the base member.

### Step S3: Cell Seeding

A cell suspension prepared to have a predetermined concentration is placed in a cell bottle in advance in a safety cabinet, and the cell bottle is installed in the closed-system cell culture device. The closed-system cell culture device feeds the cell suspension from the cell bottle to each cell culture container, and performs cell seeding. It is sufficient if the cell suspension is fed in a liquid amount enough to cover the frame body.

The cell seeding may be manually performed in step S2. In this case, the cell suspension is manually put into the cell culture container of the closed-system flow path in the safety cabinet.

As described above with reference to FIG. 1B, since the height of the frame body is sufficiently small, the height of the frame body does not affect agitation at the time of cell seeding. After the cells are uniformly dispersed in the medium in the cell culture container by agitation, the cells settle due to gravity and adhere onto the culture surface formed by the base member in the section of the frame body.

### Step S4: Culture

The cells are cultured on the base member to which the cells adhere for a predetermined time in a state in which the cell culture container is left standing. During the culture, the temperature is maintained at 37° C. by the incubator. Air in the closed-system cell culture device is always stirred by a fan so that temperature distribution is always uniform. A particle counter or a viable cell count measurement device is attached to the closed-system cell culture device, and it is possible to improve production safety by monitoring a cleanliness level.

In a case where the cell culture container including the gas permeable membrane, the filter, or the like is used for gas exchange, the gas phase in the incubator and the gas phase in the cell culture container are subjected to gas exchange via the gas permeable membrane, the filter, or the like. In a case where the cell culture container including a gas feeding flow path is used, gas exchange is performed by directly supplying predetermined gas into the cell culture container. The gas exchange is performed at a frequency of about several times a day during a culture period.

As an example, air containing 5% CO2 is used as the gas to be supplied. A flow rate of the gas is controlled from a gas cylinder to each cell culture container by a gas flow meter, and the gas is supplied in a state of being saturated with water molecules through a humidification bottle. Unnecessary gas after the gas feeding to the cell culture container is discharged to the outside of the flow path through the filter. The filter adjusts a pressure in the flow path as necessary. For example, a filter having a quality that does not pass particles of 0.62 μm or more is used as the filter.

### Step S5: Monitoring

Information regarding a cell state is acquired for the cell sheet that is being cultured by using an observation mechanism. The observation mechanism analyzes a cell proliferation rate, an occupancy rate, the cell state, and the like based on an acquired cell image and the culture supernatant. The information regarding the cell state can be acquired from one culture container for a plurality of cell sheets, so that the cell sheets can be efficiently cultured.

### Steps S6 and S7: Medium Replacement

In a case where it is the day when the medium is to be replaced, the medium is replaced. Medium addition in which the medium is simply added may be performed. The medium stored at 4° C. in a refrigerator is fed to a preheating bottle and preheated. The temperature may be raised while feeding the medium at a low liquid feeding speed. In the medium replacement, the old medium is first discharged from the cell culture container. It is sufficient if the medium is discharged so as to reach the height of the frame body.

After the discharge, the new medium is rapidly supplied into the cell culture container. The old medium is finally discharged into the culture supernatant bag. The culture supernatant in the culture supernatant bag is collected as necessary, and a growth state of the cells is evaluated by analyzing medium components such as glucose, lactic acid, and pH.

The medium replacement may be performed by a method of introducing the new medium in a state in which the old medium is contained to push out the old medium, or only the medium addition in which the new medium is supplied to the cell culture container may be performed. In addition, the closed-system cell culture device may determine whether or not to start the replacement of the medium according to a progress state of the cell culture (step S6). In a case where the replacement of the medium is not necessary (NO in step S6), the closed-system cell culture device returns to the processing of step S4. In a case where the replacement of the medium is necessary (YES in step S6), the processing proceeds to step S7, and the replacement of the medium is performed.

### Step S8: End of Cell Culture

The closed-system cell culture device determines whether or not to end the cell culture according to the progress state of the cell culture. In a case where the cell culture is to be ended (YES in step S8), the processing proceeds to step S9, the cell culture container is collected from the closed-system cell culture device, and the cell sheet is collected. In a case where the cell culture is not to be ended (NO in step S8), the closed-system cell culture device returns to the processing of step S4.

### Step S9: Collection of Cell Sheet

The cell sheet is collected according to the flow described in FIGS. 4A to 4D. The collection of the cell sheet is performed in a sterile environment. The sterile environment refers to, for example, the inside of a safety cabinet, the inside of an isolator, or the inside of a clean bench. The lid is removed, and the frame body is gripped with tweezers or the like and aseptically removed. Then, the peripheral edge of the base member having the cell sheet is gripped with tweezers or the like and aseptically taken out.

### Step S10: Packaging of Cell Sheet

According to the flow described in FIGS. 4E to 4G, the taken-out base member and cell sheet are placed on the support prepared in advance, the base member, the cell sheet, and the support are placed on the packaging member, another packaging member is put on the base member, the cell sheet, and the support so as to cover the base member, the cell sheet, and the support, and the upper and lower packaging members are thermally welded for packaging. The packaging may be performed in a state in which the base member is removed. The transportation solution is injected using the transportation solution inlet and the transportation solution outlet attached to the packaging member as necessary. The packaging has an identification code.

### Step S11: Transportation

After the packaging, the cell sheet is transported to a medical institution where transplantation is performed according to information in the identification code. An individual packaging container is transported using an appropriate transportation container capable of maintaining the temperature, pressure, and cleanliness depending on transportation means, so that quality can be maintained during the transportation.

### Step S12: Transplantation

After arrival at the operating room, the cell sheet is removed from the transportation container and aseptically removed from the packaging. At the time of opening, since there is a possibility that organisms and particles such as bacteria adhere to the outside of the individual packaging container, the individual packaging container is aseptically opened so as to maintain the cleanliness. After the opening, the base member is removed from the cell sheet by the method described in FIG. 4H. In a case where the support is a tissue adhesive, the cell sheet is transplanted together with the support. In a case where the support is a hydrophilic PVDF membrane, a nitrocellulose membrane, or the like, the hydrophilic PVDF membrane, the nitrocellulose membrane, or the like is removed after the transplantation.

### Step S13: End

In the closed-system cell culture device, the closed-system flow path used for culture is removed, various types of software of the closed-system cell culture device are terminated to end the operation of the closed-system cell culture device.

In the closed-system cell culture device configured as described above and the cell culture container used in the closed-system cell culture device, the height of the frame body is smaller than the height of the side wall of the cell culture container, so that a plurality of cell sheets can be simultaneously and automatically cultured in the closed-system cell culture device without being affected by the frame body. In addition, after the culture, the cell sheet can be taken out from the cell culture container without being peeled off. In addition, according to the present embodiment, a process of cutting the cell sheet to a size to be used for transplantation becomes unnecessary, and as a result, production efficiency is improved, and production cost can be reduced.

The frame body and the base member may be repeatedly used by being sterilized.

### Second Embodiment

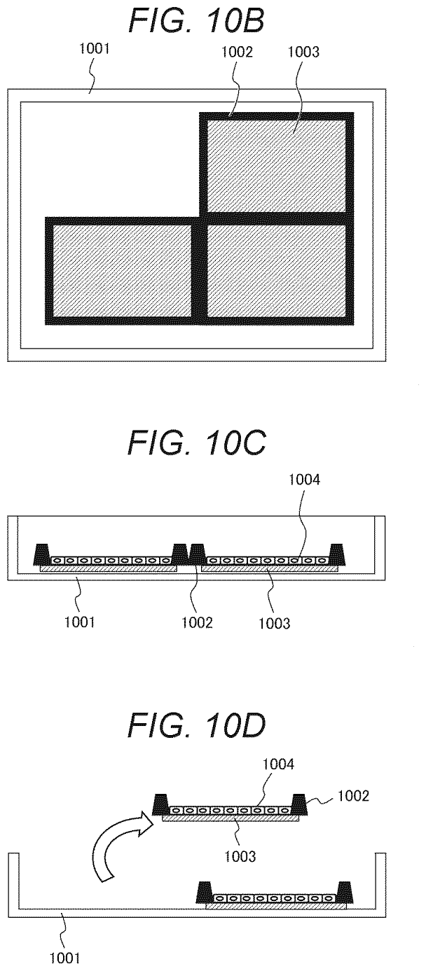

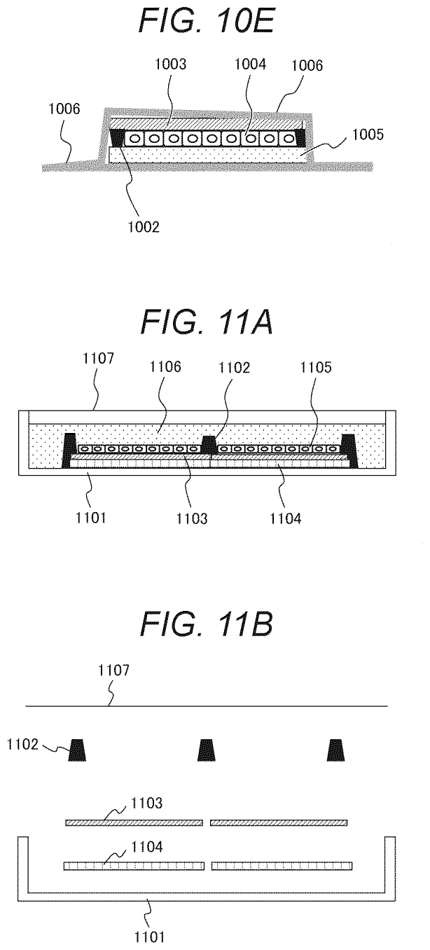

A cell culture container and a cell culture method according to a second embodiment of the present invention will be described with reference to FIGS. 9A to 10E. In the present embodiment, embodiments of a frame body and a base member different from those of the cell culture container according to the first embodiment will be described. Hereinafter, differences from the first embodiment will be mainly described.

As illustrated in FIGS. 9A to 9E, a cross-sectional shape of a frame body 901 may be a triangular shape, a T-shape, a quadrangular shape, or an inverted trapezoidal shape in addition to a trapezoidal shape, or may be a shape obtained by combining a plurality of such figures. Since an outer periphery of the frame body 901 changes based on the cross-sectional shape of the frame body 901, in a case where a frame body having a long outer periphery is selected, it is possible to prevent cells adhering to the frame body from moving to other sections. Further, in a case where an upper side of the cross-sectional shape of the frame body 901 is longer than a lower side of the cross-sectional shape of the frame body 901 as in the T-shape in FIG. 9B or the inverted trapezoidal shape in FIG. 9C, it is possible to prevent the cells from getting over the frame body.

As illustrated in FIGS. 9D and 9E, a recessed portion may be provided at the center of the frame body 901. The recessed portion provided at the center of the frame body 901 serves as an obstacle when the cells adhering onto the frame body 901 move to other sections.

As illustrated in FIGS. 10A and 10B, a frame body 1002 may be configured to be separable for each section. One base member 1003 is surrounded by one frame body 1002, and cells are cultured in the section. FIG. 10A illustrates a state during culture, and FIG. 10B illustrates a state in which one frame body 1002 is removed after the culture.

FIGS. 10C and 10D illustrate states in which FIGS. 10A and 10B are viewed from the side, respectively. One frame body 1002 may be removed as illustrated in FIG. 10D, and the frame body 1002, the base member 1003, a cell sheet 1004, and a support 1005 may be packaged in packaging members 1006 as illustrated in FIG. 10E.

In FIG. 10E, since the cultured cell sheet 1004 is surrounded by the frame body 1002, the cell sheet including the frame body can be packaged and transported, so that the cell sheet can be protected by the frame body 1002 even in a case where the cell sheet is subjected to mechanical pressure, impact, or the like during transportation.

The base member 1003 may be made of plastic as in the first embodiment, and may have a flat plate shape. In addition, the flat-plate-shaped base member 1003 becomes cell-adhesive by surface treatment or the like in advance. In the case of the flat-plate-shaped base member 1003, it is possible to prevent the base member from being rounded or prevent the base member from floating in a medium. When peeling off the cell sheet 1004, the flat-plate-shaped base member 1003 and the cell sheet 1004 are removed by being gripped with tweezers or the like.

Further, the cell sheet 1004 may be cultured on the sheet-like base member 1003 obtained by covering the inside of the section with a tissue adhesive. In a case where the base member 1003 is a tissue adhesive, the tissue adhesive and the cell sheet can be taken out from the cell culture container, and then the cell sheet 1004 can be transplanted to a patient as it is without being peeled off from the base member 1003.

### Third Embodiment

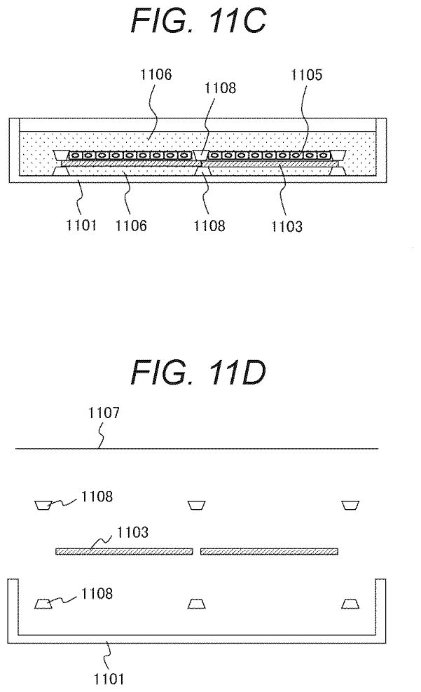

A cell culture container and a cell culture method according to a third embodiment of the present invention will be described with reference to FIGS. 11A to 11D. In the present embodiment, embodiments of a frame body and a base member different from the bottom surface of the cell culture container according to the first embodiment will be described. Hereinafter, differences from the first embodiment will be mainly described.

As illustrated in FIGS. 11A and 11B, the cell culture container of the present embodiment is different from that of the first embodiment in that a scaffold member 1104 is provided between a base member 1103 and a cell culture container 1101. Examples of the scaffold member 1104 include alginic acid, collagen gel, laminin, and a tissue adhesive.

During culture, a medium infiltrates the scaffold member 1104. Therefore, the medium is also supplied from below to cells on the base member 1103. Such a configuration makes it possible to have a favorable influence on growth of the cells. Further, the base member 1103 may be a film having a large number of pore membranes which are fine pores.

FIGS. 11C and 11D illustrate a configuration in which a frame body 1108 serving as a foot portion is provided between a bottom surface of the cell culture container 1101 and the base member 1103. As illustrated in FIG. 11C, the base member 1103 is sandwiched by the frame bodies 1108 from above and below, and the upper and lower frame bodies 1108 are positioned on the bottom surface of the cell culture container 1101 in a vertical direction, so that a medium 1106 can be provided between the base member 1103 and the cell culture container 1101. With such a configuration, the cells on the base member 1103 are supplied with the medium also from below, which has a favorable influence on the growth of the cells.

As described above, with the cell culture containers according to the first to third embodiments described above, the cultured cell sheet can be taken out without being peeled off from the base member. In addition, even in the closed-system cell culture device, it is possible to culture the cell sheet in the cell culture container having the sections, and production efficiency for the cell sheet is improved.

Note that the present invention is not limited to the above-described embodiments, but includes various modified examples and equivalent configurations within the scope of the appended claims. For example, each of the above-described embodiments has been described in detail in order to explain the present invention in an easy-to-understand manner, and the present invention is not necessarily limited to those having all the configurations described. Further, a part of the configuration of one embodiment may be replaced with the configuration of another embodiment. In addition, the configuration of another embodiment may be added to the configuration of one embodiment. In addition, a part of the configuration of each embodiment may be added with another configuration, may be deleted, or may be replaced with another configuration.

### REFERENCE SIGNS LIST

101 cell culture container
102 frame body
103 base member
104 outside portion of frame body
105 lid
106 medium
107 cell sheet
201 lid
301 cell culture container
302 frame body
303 base member
304 outside portion of frame body
401 cell culture container
402 frame body
403 base member
404 cell sheet
405 medium
406 lid
407 support
408 packaging member
501 cell culture container
502 control device
503 incubator
504 temperature adjustment unit
505 gas supply unit
506 gas concentration adjustment unit
507 pump
508 CO2/O2 sensor
509 display screen
511 medium bag/culture supernatant bag
550 temperature sensor
601 closed-system cell culture device
602 first container
603 second container (cell culture container)
604 filter
605 air pressure adjustment pipe
606 filter
607 air pressure adjustment pipe
608 first liquid feeding pipe
609 second liquid feeding pipe
610 first liquid feeding pump
611 first valve
612 filter
613 second valve
614 third container
615 filter
616 air pressure adjustment pipe
617 first discharge pipe
618 second discharge pipe
619 second liquid feeding pump
620 third valve
621 fifth valve
622 fourth container
623 sixth valve
624 eighth valve
625 air pressure adjustment pipe
626 seventh valve
901 frame body
902 base member
1001 cell culture container
1002 frame body
1003 base member
1004 cell sheet
1005 support
1006 packaging member
1101 cell culture container
1102 frame body
1103 base member
1104 scaffold member
1105 cell sheet
1106 medium
1107 lid
1108 frame body

1. A cell culture container comprising:
a frame body that partitions the culture container; and
a base member that is disposed on a bottom surface of the culture container for each of a plurality of sections partitioned by the frame body,
wherein the base member is removable for each of the sections.

2. The cell culture container according to claim 1, wherein the frame body is in contact with a peripheral edge of the base member in a vertical direction.

3. The cell culture container according to claim 1, wherein the frame body presses a peripheral edge of the base member from above.

4. The cell culture container according to claim 1, wherein the frame body is non-cell-adhesive.

5. The cell culture container according to claim 1, wherein the culture container is non-cell-adhesive.

6. The cell culture container according to claim 1, wherein the base member is a thin film having porosity.

7. The cell culture container according to claim 1, wherein a height of the frame body is smaller than a height of a side wall of the culture container.

8. The cell culture container according to claim 1, further comprising a lid that covers an opening of the culture container.

9. The cell culture container according to claim 1, wherein the frame body has a plurality of the sections.

10. The cell culture container according to claim 1, wherein a cross section of the frame body has a shape in which an upper side is longer than a lower side or a shape having a recessed portion at an upper portion of the frame body.

11. The cell culture container according to claim 1, wherein the base member is a flat-plate-shaped plastic.

12. The cell culture container according to claim 6, wherein a scaffold member is provided between the bottom surface of the culture container and the thin film.

13. The cell culture container according to claim 11, wherein a foot portion is provided between the bottom surface of the culture container and the base member.

14. The cell culture container according to claim 1, wherein the base member is a sheet-like tissue adhesive.

15. A cell culture device comprising:
a cell culture container that includes a frame body that partitions the culture container, and a base member that is disposed on a bottom surface of the culture container for each of a plurality of sections partitioned by the frame body, the base member being removable for each of the sections, the frame body having a height smaller than a height of a side wall of the culture container;
a lid that covers an opening of the culture container;
a liquid holding portion that stores a culture solution;
a liquid feeding pipe that penetrates through the lid and supplies the culture solution from the liquid holding portion to the culture container; and
a discharge pipe that penetrates through the lid and discharges the culture solution from the culture container.

16. A cell culture method comprising:
supplying a culture solution containing cells to a culture container and culturing a cell sheet on a base member that is disposed in each of a plurality of sections partitioned by a frame body and is removable for each of the sections;
removing the frame body; and
acquiring the base member on which the cultured cell sheet is placed.
